(19) United States
(12) Patent Application Publication
(10) Pub. No.: US 2022/0208310 A1
(43) Pub. Date: Jun. 30, 2022

(54) ADAPTING A RECIPE

(71) Applicant: BSH Hausgeraete GmbH, Muenchen (DE)

(72) Inventors: Daniel Gerber, Regensburg (DE); Magdalena Gorska, Warsaw (PL); Erik Kordt, Pucheim (DE); Cristina Rico Garcia, Ottobrunn (DE); Krzysztof Szporer, Warsaw (PL)

(21) Appl. No.: 17/606,122

(22) PCT Filed: Apr. 22, 2020
(86) PCT No.: PCT/EP2020/061198

§ 371 Date: Oct. 25, 2021

(30) Foreign Application Priority Data
Apr. 23, 2019 (DE) 10 2019 205 775.6

(51) Int. Cl. G16C 20/10; G16H 20/60; A23L 5/00

(52) U.S. Cl. CPC G16C 20/10; A23L 5/00; G16H 20/60

(57) ABSTRACT

A recipe contains at least one instruction for preparing an assigned quantity of at least one ingredient, in order to prepare a dish. A method for adapting the recipe includes the following steps: registering the at least one ingredient; determining a food chemical function of the ingredient; replacing the ingredient in the recipe with an alternative ingredient; and adapting the recipe such that the food chemical function that has been determined is fulfilled by at least one ingredient of the recipe.

The invention relates to the adaptation of a recipe. In particular the invention relates to the replacement of an ingredient in a recipe with another ingredient.

A recipe generally comprises a list of ingredients to be used, with each ingredient being assigned an—at least roughly estimated—quantity. The recipe also generally comprises a series of instructions for processing the specified ingredients step by step and bringing them together to make a dish.

U.S. Pat. No. 9,870,550 B2 describes a technique for extending a predetermined recipe to include an ingredient. To this end the recipe and the ingredient to be added are determined and the adaptation takes place based on known combinations of ingredients that go well together.

In a household context a dish can be prepared based on the recipe. It may be desirable for various reasons to swap one of the ingredients for another. For example a dairy product can be replaced with a soya product to take into account the lactose intolerance of someone eating the dish. In another instance it is possible to respond to someone's allergy or intolerance in respect of the ingredient. A sufficient quantity of the ingredient may not be available for preparation. The ingredient can also be swapped to alter an undesirable color, consistency, odor or taste. The ingredient can also be replaced to remove a specific substance contained therein such as alcohol or gluten.

An experienced cook can easily replace one ingredient with another without changing the character of the dish. However if an unsuitable alternative ingredient is selected, it may be difficult to monitor a cooking process or a component of the alternative ingredient together with a component of another ingredient included in the recipe may produce an unpleasant result, with the result that the dish may not be palatable.

In order to assist someone preparing the dish, particularly in a household context, a technique is required to help with the replacement of one ingredient in a recipe with another. Until now it has not been possible to harness the knowledge and experience of a good cook with technology. A knowledge-based system is complex and difficult to set up. A neural network may require an unrealistically large number of training exercises for reliable results.

It is one object of the present invention to set out an improved technique for replacing one ingredient in a recipe with another. The invention achieves this object with the subject matter of the independent claims. Preferred embodiments will emerge from the subclaims.

A recipe comprises at least one instruction for processing an assigned quantity of at least one ingredient to prepare a dish. According to a first aspect of the invention a method for adapting the recipe comprises the steps of registering the at least one ingredient; determining a food chemistry function of the ingredient; replacing the ingredient in the recipe with an alternative ingredient; and adapting the recipe such that the food chemistry function that has been determined is fulfilled by at least one ingredient included in the recipe.

Unlike with known techniques a food technology function of the ingredient to be replaced can be taken into account, thereby reliably ensuring success when preparing the dish. The food technology function is preferably determined in relation to a treatment of the ingredient or a combination with a different ingredient determined by the recipe. In one embodiment the ingredient can also be replaced with a combination of multiple ingredients.

For example cows' milk can be replaced with almond milk in a first recipe, if the sweet aroma of milk is important for the dish. In a second recipe the cows' milk can be replaced with soya or oat milk, for example if it is to be foamed. A person can determine a good replacement even without any deeper knowledge of the ingredient to be replaced. The dish can be pleasing despite the replaced ingredient. In one embodiment of the technique proposed here multiple ingredients can each be replaced with alternative ingredients, in particular one after the other.

The ingredient may fulfil a further food technology function and the recipe can be adapted such that the further function is suppressed. For example the acid from a citrus fruit can cause the protein in a milk dish to coagulate. To prevent this, for example because the quantity of citrus fruit is to be increased or because an increased tendency to flocculation is of concern in a prevailing climate, the citrus fruit can be replaced with a different fruit that contains less acid.

The food technology function of an ingredient can be enhanced or reduced as required by appropriate replacement with an alternative ingredient. In principle a larger number of food technology functions of an ingredient can be considered so that the alternative ingredient can be determined more effectively in order to ii influence several of the functions in a desired manner.

The ingredient in the recipe may fulfil multiple food technology functions and at least one function to be fulfilled by the adapted recipe or to be suppressed by the adapted recipe is registered. In other words a user can be asked to determine which of the food technology functions the ingredient in the recipe helps bring about they wish to enhance or reduce. Said user can also select multiple functions and specify what manner of influence is required.

A quantity is preferably determined for the alternative ingredient based on the food technology function and the quantity assigned to the original ingredient. The quantity of the alternative ingredient can be different from the quantity of the ingredient, for example if fresh yeast is to be replaced with dried yeast in a baking recipe.

In a further embodiment the assigned quantity of at least one further ingredient in the recipe is changed. In the above example the quantity of sugar to be used can be increased or reduced to assist the yeast fermentation process more effectively.

The adaptation can also comprise a change to an instruction included in the recipe. This can relate in particular to a series of actions to be performed, a preparation, resting or cooking time, a temperature or mechanical processing.

For example if the alternative ingredient has a shorter cooking time than the original, it can be added a specific time after the start of cooking for other ingredients.

The food chemistry function can in particular comprise a modification of at least one substance contained in an ingredient. The modification can comprise for example gelling, flocculation, binding, emulsification, coagulation or the control of a pH value, viscosity or cooking process. The food technology function preferably relates to the modification of a protein, more preferably subject to the action of heat.

The modification can generally convert or transform the substance to another. ii This can be the result of a chemical reaction or a physical conversion. A chemical reaction generally refers to a process in which one or more chemical elements or compounds are converted to different ones, with energy generally being released or absorbed. A chemical reaction is generally associated with changes to the chemical bonds in elements, molecules or crystals. A chemical reaction can cause the properties of products to change compared with their educts. A food chemistry function relates to a chemical reaction of substances that takes place during the processing of foods. The food technology function of a substance can in particular be the capacity of a substance contained in a food to take part in a predetermined chemical reaction or to facilitate the reaction.

In one preferred embodiment the food chemistry function of the ingredient and/or the alternative ingredient is determined based on a taxonomy. The taxonomy can include a grouping or categorization of the ingredients relating to one or more factors. This allows an alternative ingredient to be found more easily for an ingredient, said alternative ingredient behaving in a similar manner in respect of one or more predetermined factors. The factors can correspond for example to a physical, olfactory, aromatic or color property, a food technology function or seasonal availability.

In a further embodiment at least one alternative ingredient is determined, which can be used to replace the predetermined ingredient. This determination can be performed in particular based on the taxonomy. For example a user can specify which ingredient of a recipe they wish to replace, whereupon one or more alternative ingredients can be determined, which can be swapped for the ingredient. It can also be determined what quantity of the alternative ingredient is required or whether (and if so optionally also in what manner) an action has to be changed during preparation. The user can select one of the alternative ingredients to adapt the recipe.

In some embodiments the alternative ingredient, its quantity, the quantity of one of the other ingredients or one of the instructions is adapted based on an ontology of the ingredients. The ontology can comprise inference rules for conclusions and/or integrity rules for ensuring its validity. The ontology allows associations between ingredients to be better represented. The ontology can be established with manageable outlay and can offer adequate insight into associations during the preparation of a dish. Adaptation of the recipe can be achieved more effectively using the ontology. In particular what is known as a reasoner can be used, which derives implicit knowledge from the formalized explicit knowledge of the ontologies, in order to adapt the recipe specifically, automatically and without human intervention.

According to a further aspect of the invention a device for adapting a recipe cited herein comprises an interface for registering the at least one ingredient; and a processing facility. The processing facility here is designed to determine a food chemistry function of the ingredient; to replace the ingredient in the recipe with an alternative ingredient; and to adapt the recipe such that the food chemistry function that has been determined is fulfilled by at least one ingredient included in the recipe.

The processing facility can be designed to perform a method described herein wholly or partially. To this end the processing facility can comprise a programmable microcomputer or microcontroller and the method can be in the form of a computer program product with program code means. The computer program product can also be stored on a computer-readable data medium.

According to yet another aspect the invention comprises an electronic kitchen assistant to assist a user with cooking. Said kitchen assistant comprises the device described above, an output unit for outputting the recipe (e.g. a screen or projector), an input unit for receiving a request to replace an ingredient with an alternative ingredient (e.g. a touch screen or camera for recognizing user gestures) and a communications unit for requesting information from a central point on the internet. Food chemistry functions of ingredients for example can be requested.

Feature or advantages of the method can be applied to the device or kitchen assistant or vice versa.

The invention is described in more detail in the following with reference to the accompanying figures, in which:

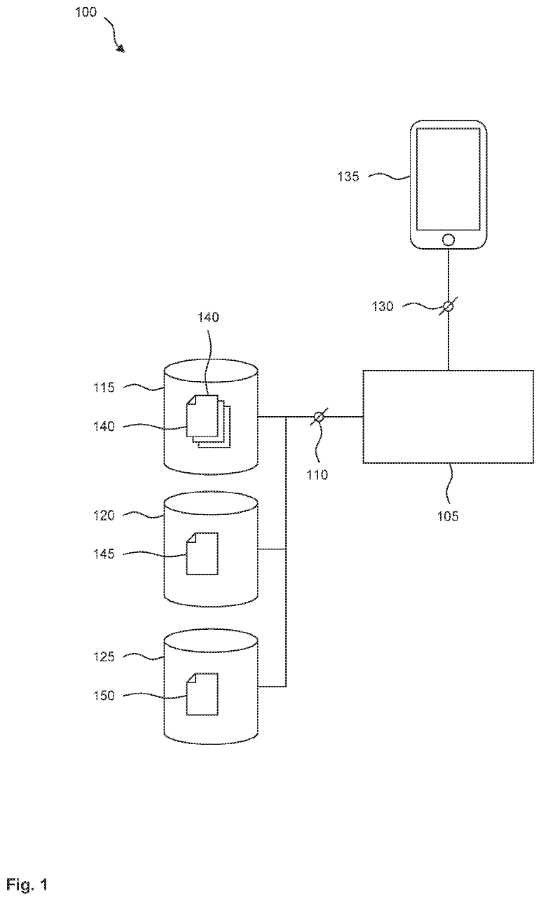

FIG. 1 shows an exemplary device;

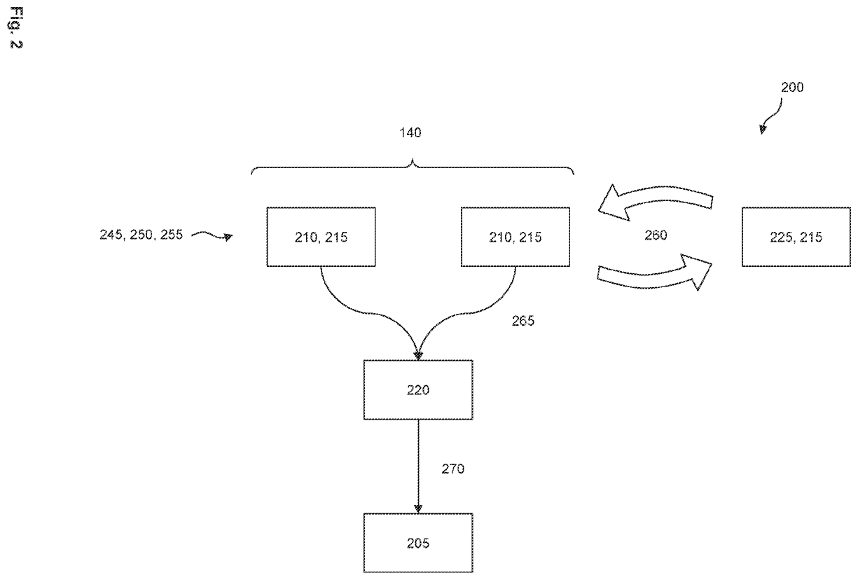

FIG. 2 shows a flow diagram of an exemplary method; and

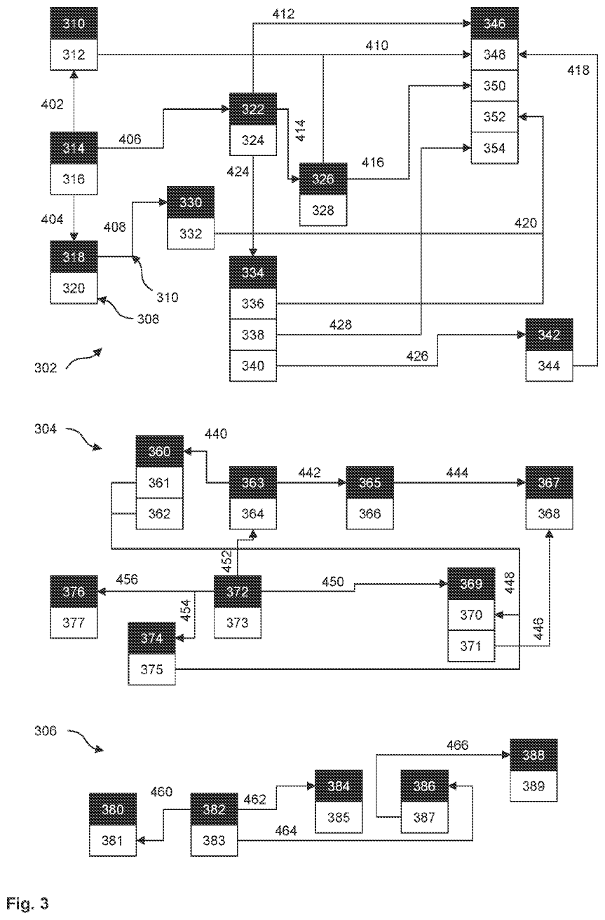

FIG. 3 shows exemplary ontologies.

FIG. 1 shows a device 100 for adapting a recipe. The device 100 preferably comprises a processing facility 105, which can be connected by means of a first interface 110 to a first storage unit 115, a second storage unit 120 and/or a third storage unit 125. Two or more of the storage units 115-130 can be integrated with one another and each storage unit 115-130 can also be assigned a dedicated first interface 110. The processing facility 105 can be connected to an interaction facility 135 by means of a second interface 130.

The first storage unit 115 is preferably designed for the storage of one or more recipes 140, which are described in more detail below, for example with reference to FIG. 2. The first storage unit 115 can also be located remotely from the device 100 and be connected for example by means of a network. The network can comprise for example the internet, a private network or a mobile radio network. The first interface 110 can be connected to the network so that the processing facility 105 has access to the recipe 140.

The second storage unit 120 is preferably designed for the storage of a taxonomy 145 of ingredients. An ingredient can be a component of a dish, which can be prepared based on the information in a recipe 140, as set out in more detail below. The taxonomy 145 can allow a grouping or classification of ingredients in respect of one or more predetermined criteria. Thus one or more different ingredients can be found for a predetermined ingredient, being in the same class in respect of a predetermined criterion or being similar to the predetermined ingredient in a predetermined manner. In a further embodiment ii the taxonomy 145 can also be organized hierarchically. The taxonomy 145 can also assist a determination of the similarity or class of an ingredient in respect of multiple criteria.

A taxonomy 145 can for example divide different fruits into botanical classes in respect of a first criterion, into different color classes in respect of a second criterion or into classes of similar cooking times or recommended cooking temperatures in respect of a third criterion. The taxonomy 145 can be organized as a multidimensional table or as a network of inputs allowing a dynamic determination of classes as a function of a search. The taxonomy here is preferably optimized with regard to request efficiency and/or required storage. In one variant the second storage unit 120 can also be arranged remotely from the device 100 and be connected to the processing facility 105 for example by means of a network and the first interface 110.

The third storage unit 125 is preferably designed for the storage of an ontology 150. The ontology 150 establishes relationships between terms and is organized such that a framework of terms can be displayed. The third storage unit 125 can also be arranged remotely from the device 100 and be connected to the processing facility 105 for example by means of a network and the first interface 110.

The interaction facility 135 is preferably designed for interaction with a person. To this end an inputting of information to the person and/or an outputting of information to the person can be assisted. The information can be exchanged optically, acoustically and/or haptically. In one embodiment the interaction facility 125 comprises a touch screen, as known for mobile telephones (smart phones). Further parts of the device 100 can be covered by the telephone or smart phone, in particular one or more of the storage units 115-125 and/or the first interface 100, which can in particular comprise a wireless communications facility, for example for the use of a mobile radio network, WLAN or Bluetooth.

In a further embodiment the interaction facility 135 comprises a projector for projecting information onto a surface, in particular a work surface or wall. Such a variant is known as a projection and interaction (PAI) facility, which can comprise a projector for projecting an image onto the surface and a scanning facility for determining a position of an object, in particular a hand of the user, in relation to the projected image. The user can thus influence for example a projected display of a recipe 140 interactively. A further variant is known as a digital assistant (Mykie), a device which can be positioned on a work surface for example and which can assist interaction by means of gestures, mimicry or verbal expression. Further parts of the device 100 can also be integrated with the interaction facility 135 in such variants.

The interaction facility 100 is designed for use in a household context, in particular in a kitchen. The interaction facility 100 allows a user preferably to select, adapt and more preferably display a recipe 140 such that said user can prepare a dish according to the information in the recipe 140.

FIG. 2 shows a schematic diagram of an exemplary recipe 140, based on which a method 200 is also described in the following. The recipe 140 represents an instruction for the preparation of a dish 205, which is preferably suitable for human consumption. The recipe 140 does not necessarily have to comprise a cooking operation; rather it can comprise the preparation of any dish 205, for example even a beverage, baked goods or a cooking product.

The recipe 140 generally comprises information about a number of required ingredients 210, an ingredient generally being assigned a required quantity 215. An ingredient 210 preferably comprises a food or a product that can be consumed by a human, at least some of which is later included in the dish 205. An ingredient 210 can itself be a dish 205 and can be produced using a recipe 140. For example an ingredient 210 can comprise a fruit, vegetable, spice, fat, meat, wine or stock.

The recipe 140 also preferably comprises one or more instructions 220, each of which specifies the manner in which an ingredient 210 is to be processed, treated, cooked, mixed or combined with another ingredient 210. An instruction 220 can also relate for example to the removal of an ingredient 210 or a part thereof from an intermediate product or the resting of an intermediate product. An intermediate product can be the result of a previous instruction 220. In this sense a finished dish 205 can also be seen as an intermediate product. A plurality of instructions 220 that are standard or non-standard in a kitchen is possible. The recipe 140 can also comprise a reference to working material or cooking appliances to be used to carry out an instruction 220, for example a food processor such as a mixer, an oven, cooker or microwave oven.

In a step 245 the recipe 140 can be registered for the execution of the recipe 140. In a step 250 a list of required ingredients 210 can then be created and optionally supplied to a user. The quantities 215 assigned to the ingredients 210 can also be supplied. The user can select from the ingredients 210 one that they wish to swap for an alternative ingredient 225. In a step 255 the selection of the ingredient 210 can be determined by the user.

In a step 260 one or more alternative ingredients 210 can be determined, which are suitable as replacements for the ingredient 210. One or more food technology functions of the ingredient 210 in the recipe 140 can be determined and the alternative ingredients 225 can preferably be determined such that they can fulfil at least one of the determined functions. A number of alternative ingredients 210 or combinations of alternative ingredients 210 can also be determined, from which the user can select one. The user can also specify an alternative ingredient 210 they wish to use.

Replacing the ingredient 210 with one or more alternative ingredients 225 can upset the balance of the recipe 140. For example all the intended food technology functions in the recipe 140 may no longer be fulfilled, the instructions 220 relating to ingredients 210, 225 may be incongruous or nonsensical or the original ratios of quantities of ingredients 210 may be disrupted. The recipe 140 can be adapted in order to control the impact of swapping the ingredient 210 for the at least one alternative ingredient 225. In particular a quantity 215 of a further ingredient 210 and/or an instruction 220 in the recipe 140 can be adapted. Adaptation can be performed such that at least one of the determined functions is fulfilled when the recipe 140 is implemented. A reasoner in particular can be used, which adapts the recipe automatically with the aid of the ontologies.

A further replacement of an ingredient 210 can then be made or the recipe 140 can be implemented. For implementation in a step 265 one or more ingredients 210, 225 can be processed by carrying out a corresponding instruction 220. Further processing steps can then follow with further ingredients 210, 225 and/or intermediate products. In a step 270 the desired dish 205 can be completed from the application of an instruction 220. Implementation of the recipe 140 is generally reserved for a person but can be partially or wholly automated. Preparation of the dish 205 according to the recipe 140 by a person can be assisted, in particular by means of context-sensitive provision of information relating in each instance to an instruction 220 to be implemented. A user can thus be guided or accompanied through the implementation of the recipe 140 step by step.

FIG. 3 shows graphic representations of exemplary ontologies. A first ontology 302 relates to a recipe 140, a second ontology 304 relates to a chemical property of an ingredient 210 and a third ontology 306 relates to an ingredient 210. Terms 308 are shown by boxes and relationships 310 between terms 308 are shown correspondingly by connections. The terms 308 each comprise a category (light text on dark background) and one or more instances (dark text on light background). The first ontology 302 uses terms 312-345 and relationships 402-428; the second ontology 304 terms 360-377 and relationships 440-456; the third ontology 306 terms 380-389 and relationships 460-466. An example of the adaptation of a recipe 140 is set out in the following based on the illustrated ontologies 302-306.

A recipe 140 for a dish 205 in the form of a muffin contains the ingredient 210 honey. A user preparing the muffin 205 wishes to reduce the sugar content of the muffin 205. A system 100 performing a method 200 can recommend replacing the honey 210 with stevia and mashed banana as alternative ingredients 225. Appropriate quantities 215 of the alternative ingredients 225 can ii be determined and supplied. The recommendations can be derived from chemical properties, inferred ratios and further information relating to honey 210, referred to here as food chemistry functions.

The ontologies 302-306 can be used to research chemical properties of honey 210:
“sweetness”: derived from the inherent sweetness of honey 210 and its components
“weight”: based on the significant quantity 215 of honey 210 in the recipe 140 in relation to the total quantity of all the ingredients 210 the contribution of honey cannot be ignored. A quantity 215 of honey 210 left out must be replaced with other ingredients 210, 225 to provide muffins 205 of a similar weight.
brown color: as honey 210 contains reducing sugars and a baking process 220 involves a high temperature, the Maillard reaction takes place at high speed, making the muffins 205 brown. It should be noted that this is not an inherent function of honey 210 but is only observed in a finished muffin 205 due to the heating process 220.
“moistness”: as honey 210 contains a high proportion of hygroscopic sugars, it binds moisture during baking and storage of the muffins 205.

If an ingredient 210 is to be replaced, all the food technology functions of the original ingredient 210 should be brought about where possible by one or more alternative ingredients 225.
As sweetness is a dominant property of the muffin 205, a search for an alternative ingredient 225 can start with this property.
The ingredient 210 stevia has the function of “sweetness” and can therefore possibly be used as an alternative ingredient 225 for honey 210.
The quantity 215 of stevia 225 is determined on the basis of respective sweetness indices. If for example the available stevia 225 is 30 times sweeter than honey 210, a 30 times smaller quantity 215 than of honey 210 should be used.
The other functions of honey 210 relating to the muffin 205 are not yet fulfilled. Mashed banana 225 is selected as a second alternative ingredient 225, as banana 225 can fulfil two as yet unfulfilled functions of honey 210.
Banana 225 can bring about a brown coloration as it can contain polyphenol oxidase and polyphenols. When the cell structure of the banana 225 is destroyed, these two substances react with one another and with oxygen in the air, resulting in the fruit browning reaction of the banana 225.
Banana 225 contains a lot of water and can contribute to the moistness of the finished muffin 205.
As the quantity 215 of stevia is already determined, the quantity 215 of banana 225 should be determined so that together with the quantity 215 of stevia 225 it makes up the quantity 215 of honey 210. The quantity 215 of banana 225 can be determined taking different susceptibilities of the alternative ingredients 225 to weight loss when warmed into account.

This example shows that ingredients 201, 225 can fulfil different food chemistry functions in different contexts. These can be divided into:

1. Functions determined by chemical properties of an ingredient 210, 225;

2. Functions determined by a chemical reaction of an ingredient 210, 225 with another ingredient 210, 225 or a surrounding substance; and

3. Functions determined by chemical properties in combination with external conditions. The conditions can relate in particular to a mechanical and/or thermal treatment mode.

Some of the embodiments described here can have the advantage that a user can swap some ingredients of a recipe spontaneously while cooking, for example because they are not available at the time, and it is still ensured that the user is advised by means of information technology so that the end result of the recipe will be successful. With the aid of formalized knowledge of cooking associations, for example in the form of ontologies and reasoning, the system can preferably counteract possible sources of error due to lack of competence on the part of the user.

### REFERENCE CHARACTERS

100 Device

105 Processing facility

110 First interface

115 First storage unit (recipe)

120 Second storage unit (taxonomy)

125 Third storage unit (ontology)

130 Second interface

135 Interaction facility

140 Recipe

145 Taxonomy

150 Ontology

200 Method

205 Dish

210 Ingredient

215 Quantity

220 Instruction

225 Alternative ingredient

245 Register recipe

250 Supply list

255 Determine selection of an ingredient

260 Swap ingredient, adapt recipe

265 Process

270 Complete dish

302 First ontology

304 Second ontology

306 Third ontology

308 Term

310 Relationship

312 Recipe category

314 Recipe

316 Muffin

318 Preparation mode

320 Bake

322 Has ingredient

324 Honey

326 Quantity according to recipe

328 20%

330 Temperature

332 180° C.

334 Food chemistry properties

336 Reduce sugar

338 Hygroscopic

340 Sweetness

342 Sweetness index

344 1

346 Function in finished dish

348 Sweetness

350 Weight/quantity

352 Brown color

354 Moistness

360 Food chemistry properties

361 Contains polyphenol oxidase

362 Contains polyphenols

363 Ingredient

364 Banana

365 Nutritional property

366 Water content

367 Nutritional value

368 70%

369 Ingredient function

370 Brown color

371 Moistness

372 Ingredient with state

373 Mashed banana

374 State

375 Mashed

376 Ingredient family

377 Fruit

380 Ingredient

381 Stevia

382 Ingredient with state

383 Stevia powder

384 Ingredient property

385 Sweetness

386 Food chemistry properties

387 Sweetness

388 Sweetness index

389 30

402 Has context

404 Has preparation mode

406 Has ingredient

408 Has temperature

410 Required function for present recipe

412 Has function in finished dish

414 Has quantity

416 Made from

418 Triggers function

420 Maillard reaction due to high temperature and presence of reduced sugar

424 Has food chemistry properties

426 Has sweetness index 428 Hygroscopic response binds water

440 Has food chemistry property

442 Has nutritional property

444 Has value

446 Made from

448 Brought about by combination

450 Has function

452 Has ingredient

454 Has state

456 Belongs to family

460 Has basic ingredient

462 Has function

464 Has food chemistry property

466 Has sweetness index

1-11. (canceled)

12. A method of adapting a recipe, the recipe including at least one instruction for processing an assigned quantity of at least one ingredient to prepare a dish, the method comprising the following steps:
registering the at least one ingredient;
determining a food chemistry function of the at least one ingredient;
replacing the at least one ingredient in the recipe with an alternative ingredient; and
adapting the recipe such that the food chemistry function of the at least one ingredient is fulfilled by at least one ingredient included in the recipe.

13. The method according to claim 12, wherein the at least one ingredient fulfils a further food-technological function and the adapting step comprises adapting the recipe to suppress the further food-technological function.

14. The method according to claim 12, wherein the at least one ingredient in the recipe fulfils multiple food-technological functions and the method comprises registering at least one function to be fulfilled by the adapted recipe or to be suppressed by the adapted recipe.

15. The method according to claim 12, wherein the adapting step comprises changing an assigned quantity of at least one further ingredient in the recipe.

16. The method according to claim 12, wherein the adapting step comprises changing an instruction included in the recipe.

17. The method according to claim 12, wherein the food chemistry function comprises a modification of at least one substance contained in an ingredient.

18. The method according to claim 12, which comprises determining the food chemistry function of at least one of the ingredient or the alternative ingredient based on a taxonomy.

19. The method according to claim 12, which comprises determining at least one alternative ingredient which can be used to replace the at least one ingredient.

20. The method according to claim 12, which comprises adapting an alternative ingredient, a quantity of the alternative ingredient, a quantity of any of the ingredients or one of the instructions based on an ontology of the ingredients.

21. A device for adapting a recipe, wherein the recipe comprises at least one instruction for processing an assigned quantity of at least one ingredient to prepare a dish, the device comprising the following:
an interface for registering the at least one ingredient;
a processing facility which is configured to:
determine a food chemistry function of the ingredient;
replace the ingredient in the recipe with an alternative ingredient; and
adapt the recipe such that the food chemistry function determined for the ingredient is fulfilled by at least one ingredient included in the recipe.

22. An electronic kitchen assistant for assisting a user with cooking, the kitchen assistant comprising:
a device according to claim 21;
an output unit for outputting the recipe;
an input unit for receiving a request to replace an ingredient of the recipe with an alternative ingredient; and
a communications unit for requesting information from a central point on the internet.
